(19) United States
(12) Patent Application Publication
(10) Pub. No.: US 2025/0218556 A1
(43) Pub. Date: Jul. 3, 2025

(54) METHODS FOR MAXIMUM JOINT PROBABILITY ASSIGNMENT TO SEQUENTIAL BINARY RANDOM VARIABLES IN QUADRATIC TIME COMPLEXITY

(71) Applicant: January, Inc., Menlo Park, CA (US)

(72) Inventors: Mark WOODWARD, Fresno, CA (US); Saransh AGARWAL, San Francisco, CA (US)

(21) Appl. No.: 18/958,126

(22) Filed: Nov. 25, 2024

Related U.S. Application Data
(63) Continuation of application No. PCT/US2023/023673, filed on May 26, 2023.
(60) Provisional application No. 63/347,291, filed on May 31, 2022.

(51) Int. Cl. G16H 10/60; G16H 10/40; G16H 20/30; G16H 20/60

(52) U.S. Cl. CPC G16H 10/60; G16H 10/40; G16H 20/30; G16H 20/60

(57) ABSTRACT

A method may store time series data that includes a biophysical response over sequential time periods. An initial variable can be established having an event value corresponding to each time period. A plurality of assigned variables can be generated, each having an assigned event value corresponding to each time period with one assigned event value being different with respect those of the initial variable and the other assigned variables. The initial and assigned variables can be evaluated with a probability function to determine the variable having a highest probability of event occurrences with respect to the biophysical responses. Using the highest probability initial or assigned variable as the initial variable, generation of assigned variables and a highest probability determination can be repeated until a highest probability variable has been determined. The highest probability variable can be used to predict the biophysical response in a user. Corresponding systems are also disclosed.

### RELATED APPLICATIONS

This application is a continuation of International Application No. PCT/US2023/023673, filed May 26, 2023, which claims the benefit of U.S. Provisional Application No. 63/347,291, filed May 31, 2022, each of which is incorporated by reference herein in its entirety.

### BACKGROUND

The present disclosure relates generally to the creation and use of biophysical and behavioral models using machine learning, and more particularly to methods and systems for predicting subject responses with such models.

It is often useful to know the assignment of values to a time series that is the most probable of all possible assignments. For example, a time series of blood glucose measurements for a subject can be analyzed to determine what times that subject ate meals. In the meal example, for each time step of the time series there may be inputs corresponding to a blood glucose measurement and other biomarker data, and there may be a binary random variable corresponding to whether a meal was eaten at that time step.

Continuing with the meal example, for a given input of bio marker data and an assignment to all of the binary “meal” random variables it may be possible to calculate the probability of that assignment. However, there are an exponential number of assignments to the “meal” random variables, so evaluating all assignments to find the most probable assignment may be computationally intractable. For example, if there are 100 timesteps and 1 binary “meal” variable per time step, then there are 2 to the power of 100 possible assignments, which is over 1 million trillion trillion assignments.

### SUMMARY

In one aspect, the present disclosure provides a method for predicting events from time series biological response data. A method can include storing time series data with a storage device. The time series data can include one or more biophysical responses over sequential time periods. By operation of a computing device, an initial variable can be established having an event value corresponding to each time period of the time series. A plurality of assigned variables can be generated, each having an assigned event value corresponding to each time period of the time series. Each assigned variable can have one assigned event value being different with respect those of the initial variable and the other assigned variables. The initial and assigned variables can be evaluated with a probability function to determine the initial or assigned variable having a highest probability of event occurrences with respect to the biophysical responses in the time periods. Using the highest probability initial or assigned variable as the initial variable, the generation of assigned variables and evaluation of the initial and assigned variables can be repeated until a highest probability initial or assigned variable has been determined. A highest probability initial or assigned variable can be used to predict the at least one biophysical response in a user.

In an aspect, a method is disclosed, comprising: storing time series data with a storage device, the time series data including at least one biophysical response over sequential time periods; by operation of a computing device establishing an initial variable, having an event value corresponding to each time period, generating a plurality of assigned variables, each having an assigned event value corresponding to each time period with one assigned event value being different with respect those of the initial variable and the other assigned variables, evaluating the initial and assigned variables with a probability function to determine the initial or assigned variable having a highest probability of event occurrences with respect to the biophysical responses in the time periods, and using the highest probability initial or assigned variable as the initial variable, repeating the generating the assigned variables and evaluating of the initial and assigned variables until a highest probability initial or assigned variable has been determined; and using the highest probability initial or assigned variable to predict the at least one biophysical response in a user.

In some embodiments, the method of claim 1, wherein the time series data comprises blood glucose values over the sequential time periods.

In some embodiments, the event value corresponds to a meal.

In some embodiments, the event value corresponds to physical activity

In some embodiments, the initial variable is a binary value, with each event value corresponding to a bit location of the binary value, each bit location corresponding to each time period of the time series; and each assigned variable is a binary value, with each assigned event value corresponding to a bit location of the binary value, each bit location corresponding to each time period of the time series.

In some embodiments, establishing the initial variable includes setting all bits of the initial variable to a value indicating no event, and generating the assigned variables includes changing a different bit in each assigned variable a value indicating an event.

In some embodiments, evaluating the initial and assigned variables with a probability function comprises applying the initial and assigned variables to a probability prediction statistical model to generate a probability value for each initial and assigned variable.

In some embodiments, the method further includes training a statistical model with training sets of time series data of the least one biophysical response and corresponding events to generate the probability prediction statistical model.

In some embodiments, evaluating the initial and assigned variables with the probability function includes determining a conditional probability for one time period using conditional probabilities for all previous time periods.

In some embodiments, the computing device comprises a plurality of parallel processing paths, each processing path configured to determine a probability for a different initial or assigned variable, and a variable selector coupled to each processing path and configured to select the highest probability initial or assigned variable.

In some embodiments, the computing device comprises a hardware accelerator unit selected from the group of: a graphics processing unit (GPU) and tensor processing unit (TPU); and the parallel processing paths are within the GPU or TPU.

In an aspect, a system is disclosed, comprising: data storage configured to store time series data that included at least one biophysical response over sequential time periods; and at least one computing device coupled to the data storage can configured to establish an initial variable, having an event value corresponding to each time period of the time series, generate a plurality of assigned variables, each having an assigned event value corresponding to each time period of the time series, one assigned event value being different with respect those of the initial variable and the other assigned variables; evaluating the initial and assigned variables with a probability function to determine the initial or assigned variable having a highest probability of event occurrences with respect to the biophysical responses in the time periods, using the highest probability initial or assigned variable as the initial variable, repeating the generating the assigned variables and evaluating of the initial and assigned variables until a highest probability initial or assigned variable has been determined, and using the highest probability initial or assigned variable to predict the at least one biophysical response in a user.

In some embodiments, the at least one computing device comprises at least one statistical model configured to generate probability values for received initial variables and assigned variables.

In some embodiments, the at least one statistical model comprises at least one artificial neural network (ANN).

In some embodiments, the at least one ANN comprises a recurrent ANN.

In some embodiments, the at least one recurrent ANN is selected from the group of: a long short-term memory and gated current unit.

In some embodiments, the at least one computing device comprises at least one hardware accelerator unit having a plurality of processing paths, each processing path configured to determine the probability of the initial variable or one of the assigned variables.

In some embodiments, the at least one hardware accelerator is selected from the group of: a graphics processing unit and a tensor processing unit.

In some embodiments, the time series data comprises a blood glucose values over the sequential time periods.

In some embodiments, the event value is selected from the group of: a meal and a physical activity.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A is a block diagram of a system according to an embodiment;

FIG. 1B is a block diagram of a system and method according to an embodiment;

FIG. 2A is a block diagram of a health behavior model (HBM) system and method according to an embodiment;

FIG. 2B is a block diagram of a HBM system and method according to another embodiment;

FIGS. 2C-0 to 2C-2 are block diagrams showing systems and methods for generating an optimal messaging approach for a user according to embodiments;

FIG. 3A is a block diagram of a method and system for generating a latent space representation of user lifestyle according to embodiments;

FIG. 3B is a block diagram of a method and system for generating a latent space representation of user lifestyle according to embodiments;

FIGS. 4A-0 and 4A-1 are diagrams showing systems and methods for generating food recommendations for users using collaborative filtering according to embodiments;

FIGS. 4B-0 and 4B-1 are diagrams showing systems and methods for generating food recommendations for users using collaborative filtering according to additional embodiments;

FIG. 5 is a block diagram of a method and system for generating synthetic user models having adherence responses to recommendations according to embodiments;

FIGS. 6A-0 to 6A-2 are diagrams showing methods and systems for generating realistic biophysical responses with a generative adversary network (GAN) according to embodiments;

FIGS. 6B-0 and 6B-3 are diagrams showing methods and systems for generating realistic biophysical responses with a GAN according to additional embodiments;

FIG. 7A is a block diagram of a method and system for learning non-linear and other relationships for biophysical phenomena according to embodiments;

FIG. 7B is a block diagram of a method for training a system to interpolate multi-modal sensor data according to embodiments;

FIG. 7C is a block diagram of a method and system for interpolating multi-modal sensor data according to embodiments;

FIG. 8A and 8B are a block diagrams showing systems and methods for generating food recommendations based on predetermined food features according to embodiments;

FIGS. 9A and 9B are diagrams showing systems and methods for inferring clinical responses of users based on sensor and other data according to embodiments;

FIGS. 9C and 9D are diagrams showing systems and methods for inferring clinical responses of users based on sensor and other data according to additional embodiments;

FIG. 10A is a block diagram showing a system and method for ensuring adequate recordings of users' actions based on predicted actions according to embodiments;

FIG. 10B is a block diagram showing a system and method for ensuring adequate recordings of users' actions based on predicted actions according to additional embodiments; and

FIG. 11 shows a computer system that is programmed or otherwise configured to implement methods provided herein.

FIG. 12 is a flow diagram of a method 1200 according to another embodiment.

FIG. 13 is a sequence of diagram showing variable generation and selection according to an embodiment.

FIG. 14A shows a system and corresponding methods 1400A for generating conditional probabilities according to an embodiments.

FIG. 14B shows systems and methods for generating event data according to another embodiment.

FIG. 14C shows training 1400C/I (train) which can create a function to infer the probability of an event at other time periods ti.

FIG. 15 is a diagram showing parallel processing operations of variable values according to an embodiment.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Embodiments can generate high or highest probability assignment of events to biological time series data with practical computation times and/or resources. While any suitable biological time series data can benefit from event assignments as described herein, some embodiments can assign meal event data to blood glucose time series data.

In cases such as the meal prediction problem above, where the random variables are binary and the effect of the random variable events on the measurements is additive, i.e. eating more meals only increases blood glucose levels, we introduce an iterative procedure which finds the optimal assignment in at most T\hat{ }2 assignment evaluations, where T is the number of time steps. For the example above with 100 times steps, at most 10,000 assignments would be evaluated, a reduction in processing of 26 orders of magnitude over evaluating all assignments.

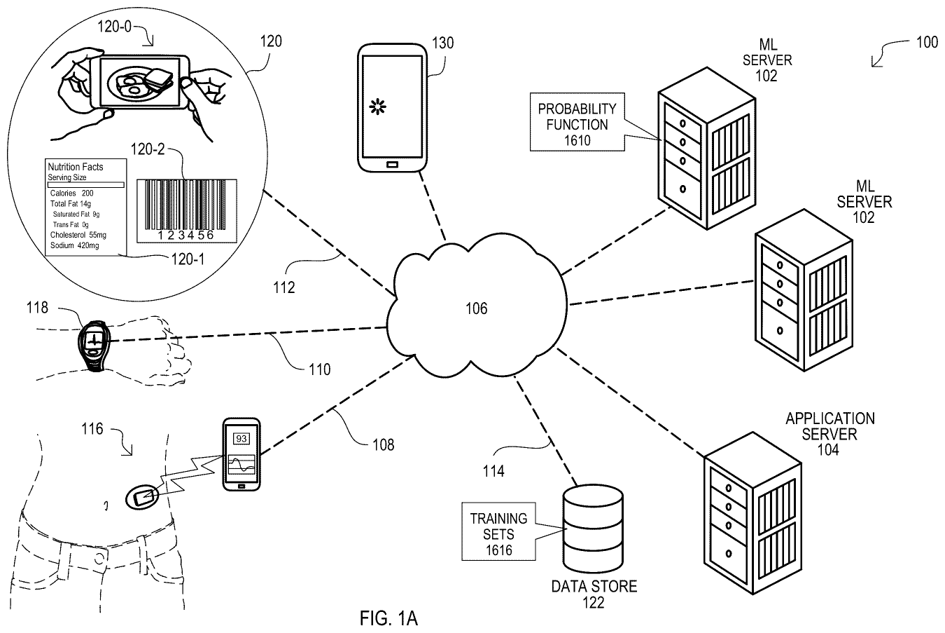

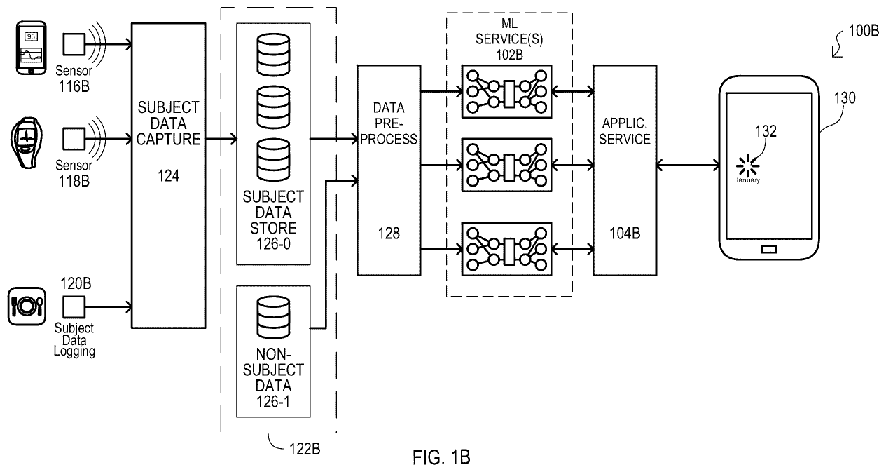

FIG. 1A is a conceptual and block diagram of a system 100 according to an embodiment. A system 100 can include one or more machine learning servers 102, application servers 104, data store 122, multiple data sources (108, 110, 112), and subject devices 130. Data sources (108, 110, 112), servers (102, 104) and subject devices 130 can be in communication with one another through a network 106, which can include various interconnected networks, including the Internet.

Machine learning (ML) servers (102/104) can include one or more statistical models, including artificial neural networks (ANN) of various architectures, and related systems, as will be described herein and equivalents. ML servers (102/104) can execute various functions, including learning (e.g., training) and inference functions according to data received from data sources (116, 118, 120) as well as other data residing on date storage 122. In some embodiments, ML servers (102/104) can include any suitable statistical learning agent, including any dimensionality reducer appropriate the domain and training data, such as autoencoders (AEs), as well as any of generative adversarial networks (GANs), long short-term memory networks (LSTMs), convolutional neural networks (CNNs), reinforcement learning (RL) algorithms, and any other ANN or related architecture suitable for the systems and methods described herein. ML servers (102/104) can also include specialized algorithms for use by various processes, including equation solving algorithms, such as differential equation (DE) solvers. In some embodiments, one or more machine learning servers 102 can include a probability function 1610 for assigning events to biological response time series data, as is described herein.

An application server 104 can interact with one or more applications running on a subject device 130. In some embodiments, data from data sources (116, 118, 120) can be acquired via one or more applications on a subject device (e.g., smart phone) and provided to application server 104. Application server 104 can communicate with subject device 130 according to any suitable secure network protocol.

ML servers 102 and application server 104 can include any suitable computing system having one or more processing devices.

A data store 122 can store data for system 100. In some embodiments, data store 122 can store data received from data sources (116, 118, 120) (e.g., from subjects) as well as other data sets acquired by third parties. Data store 122 can also store various other types of data, including ANN configuration data for configuring networks of ML servers (102/104). A data store 122 can take any suitable form, including one or more network attached storage systems. In some embodiments, all or a portion of data store 122 can be integrated with any of the servers (102, 104). In some embodiments, a data store 122 can store training sets 1616 for training one or more machine learning modes to assign a probability of an event with respect to time series biological response data, as described herein.

In some embodiments, data for data sources (108, 110, 112) can be generated by sensors or can be logged data provided by subjects. In the example shown, data source 108 can correspond to a first type sensor 116, data source 110 can correspond to a second type sensor 118, and data source 112 can correspond to logged data 120 provided from a subject. Logged data 120 can include data from any suitable source, including text data as well as image data.

According to some embodiments, a first type sensor 116 can be a “direct” data source, providing values for a biophysical subject response that can be predicted by the system 100. A second type sensor 118 and logged data 120 can be “indirect” data sources. Such “indirect” data sources can be provided as inputs to biophysical models of the system 120 to infer future a biophysical response different from the response(s) the second type sensor 118 detects and/or activity indicated by logged data 120. In some embodiments, both direct and indirect data can be used to train and calibrate biophysical models. However, in some embodiments, direct data may not be used in inference operations. In some embodiments, a first type sensor 116 can be a sensor that is more difficult to employ than a second type sensor 118. In some embodiments, sensors (116, 118) can have data captured by a subject device 130, which can then send such data to servers (102/104), such sensors (116, 118) can also transmit such data to servers without a subject device (e.g., directly, or via one or more intermediate devices).

In some embodiments, a first type sensor 116 can be a continuous glucose monitor (CGM), which can track a glucose level of a subject. A second type sensor 118 can be heart rate monitor (HRM) which can track a subject's heart rate. Logged data 120 can be subject nutrition data. In some embodiments, nutrition data 120 can be acquired by an application on a subject device 130. In some embodiments, image data can be captured, and such image data can be used as inputs to models on ML servers (102) to infer nutrition values. Image data can be images of text (e.g., labels 120-1) which can be subject to optical character recognition to generate text, and such text can be applied to an inference engine or other ontological system. In addition to or alternatively, image data can be images of actual food (e.g., 120-0), or food packaging, and such image data can be applied to an inference engine or other model to derive nutrition values. Further, logging can include capturing standardized labels (e.g., 120-2) which can be subject to a database search or ML model to derive nutrition values.

A subject device 130 can be any suitable device, including but not limited to, a smart phone, personal computer, wearable device, or tablet computing device. The subject device 130 can include one or more applications that can communicate with application server 122 to provide data to, and receive data from, biophysical models residing on ML servers 102. In some embodiments, a subject device 130 can be an intermediary for any of data sources (108, 110, 112).

Referring to FIG. 1B, a system and method 100B according to another embodiment is shown in a block diagram. A system 100B can include data source inputs 116B, 118B, 120B, a data subject capture portion 124, a storage portion 122B, a data pre-processing portion 128, a ML services portion 102B, and an application services portion 104B. Data source inputs (116B, 118B, 120B) can provide data for learning operations in ML services 102B that create biophysical models for a subject. All or a portion of data source inputs (116B, 118B, 120B) can provide data for inference operations executed on models resident in ML services portion 104B. In very particular embodiments, data source inputs (116B, 118B, 120B) can include any of the sensors and/or subject data logging described herein or equivalents.

Data store portion 122B can include subject data storage 126-0 as well as non-subject data storage 126-1. Subject data storage 126-0 can store data for particular subjects for which ML models have been created or are being created. Non-subject data storage 126-1 can include data derived from other sources that can be used for training purposes (e.g., data from non-subjects, such as studies conducted by third parties, or synthetic user data as described herein or an equivalent).

A data pre-processing portion 128 can process data from data store portion 122B for application to ML services 102B. Data pre-processing portion 128 can include instructions executable by a processor to place data into particular formats for processing by ML services 102B.

ML services 102B can include computing systems configured to create ML models through supervised and/or unsupervised learning with any of data source inputs 116B, 118B, 120B. In addition, ML services 102B can include ML models that generate inferences based on any of data source inputs 116B, 118B, 120B. ML services 102B can include single computing devices that include ANNs of the various architectures described herein, as well as ANNs distributed over networks. As in the case of FIG. 1A, ML services 102B can include AEs, GANs, LSTMs, CNNs, RL algorithms, and any other suitable ANN or other statistical learning agent, and related architecture.

Application services 104B can access models resident in ML services 102B to provide data for one or more subject applications 132. Subject applications 132 can utilize model outputs to provide information to subjects. In some embodiments, applications 132 can provide recommended actions for subjects based on subject responses predicted by models in ML services 102B. In some embodiments, applications 132 can recommend subject actions based on predicted glucose levels of subjects and/or sensor and logged data of subjects. In the embodiment shown, application services 104B can service applications 132 running on subject devices 130. However, in other embodiments application services 104B can execute applications and provide (e.g., push) data to other services (e.g., email, text, social network, etc.).

Embodiments can also include methods and systems for generating personal recommendations for a subject (e.g., user) based on a behavior model of the subject. A system can combine psychometric data with sensor and other data to provide contextual recommendation to a subject.

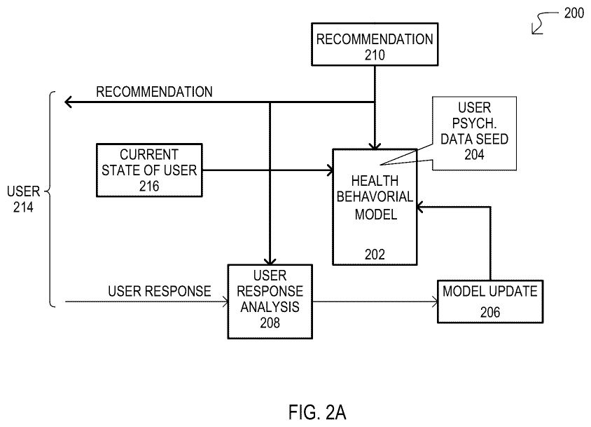

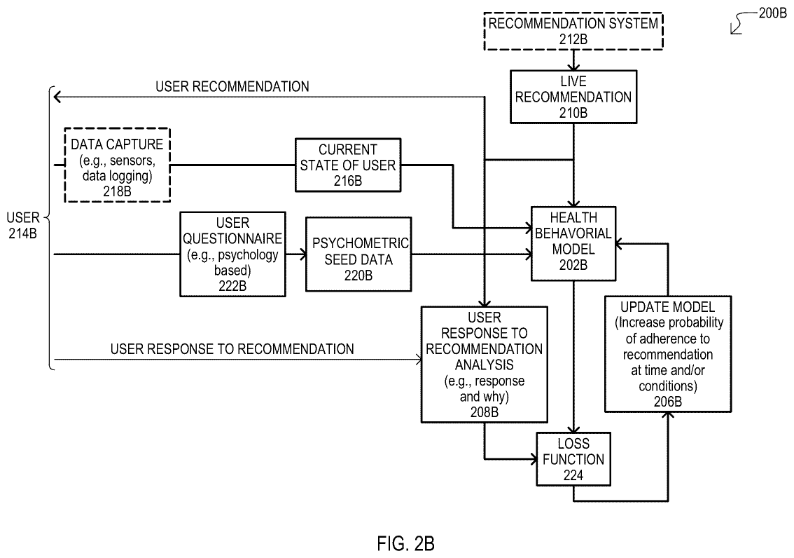

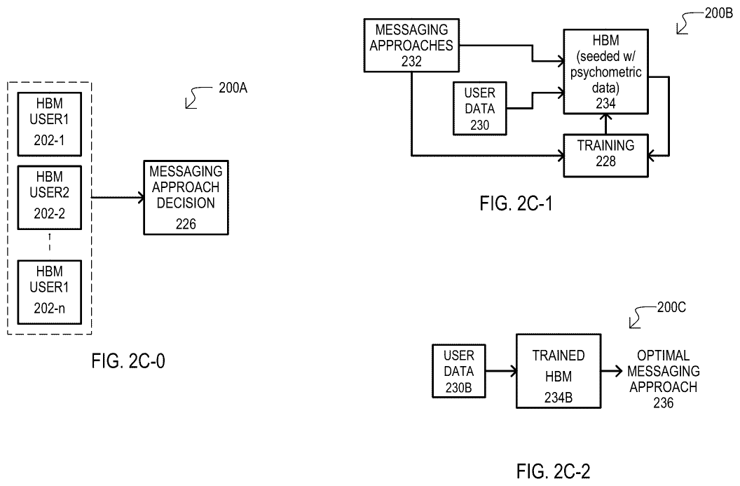

FIG. 2A shows a recommendation system and method 200 according to embodiments. A system 200 can include a health behavior model (HBM) 202, response analyzer 208, and model updater 206. A HBM 202 can include neural network or other statistical model seeded with personalized psychology-based data 204 of a user. User psychology-based data 204 can take any suitable form, including but not limited to data directly generated by a user (e.g., questionnaire answers, test results) and/or data acquired from recorded user actions. User psychology-based data 204 can take any suitable form, including numerical or string data or a latent space encoding of psychology-based data.

A response analyzer 208 can compare a user response to a recommendation and provide a result to model updater 206. A response analyzer 208 can provide any of a number of response types, from determining whether or not a recommendation has been followed, to a classification of how close a user response was to a recommendation. A model updater 206 can alter HBM 202 in response to results from response analyzer 208. A model update 206 can be suitable to the type of HBM 202. In this way, HBM 202 can be updated over time as a user responds to recommendations.

As the user makes decisions on recommendations sensor and/or other data can be aggregated and used to further train the model to learn user behavior and thus provide recommendations that more likely to be followed (have a higher adherence rate).

In operation, recommendations 210 can be received by a recommendation system 210, which may or may not be part of system 200. Recommendations can be sent to a user 214 (i.e., to a user device), and also HBM 202 and response analyzer 208. In addition, a current state of a user 216 can be received by HBM 216. A current state of a user 216 can include data that represents a user's state over a time period in which the recommendation is received.

Following the issuing of a recommendation, response analyzer 208 can receive a user response and determine an adherence by a user 214 to the recommendation. A user response can be generated by a user 214 directly (e.g., answering a query) and/or indirectly (monitoring sensor data of the user). Based on adherence data from response analyzer 208, model updater 206 can update HBM 202. Such an update operation can consider a state of user 216 when a recommendation is received, and thus learning by HBM can include contextual states related to user recommendation decisions.

In some embodiments, a trained HBM 202 can be used to infer an adherence likelihood of a subject (e.g., user) based on a recommendation and user state.

FIG. 2B shows a system and method 200B according to another embodiment. A system 200B can use psychometrics and sensor data to create a user model that can predict a user adherence to recommendations. A user 214B can provide answers to a questionnaire 222B. Data from questionnaire 222B can be organized as psychometric data 220B which can be used to seed a machine learning HBM model 202B. Accordingly, HBM 202B can be initialized with personalized psychometric data.

A system 200B can receive current user state data 216B, all or part of which can be generated data captured from a user 214B. Such data can include, but is not limited to, sensor data from sensors that can read one or more biophysical states of a user, as well as data logged by a user. In some embodiments, a system 200B can evaluate food recommendations for a user, and sensor data can include any of: glucose level data (e.g., data generated by a), heart rate data (e.g., data from an HRM), and position data (e.g., position data generated by user device, such a location data from a cellular communication system and/or global positioning system, GPS). Logged data in such a system can be food log data (i.e., a record of food eaten by a user).

Seeded with psychometric data, and receiving current user state data (e.g., state data current with an issued recommendation) 216B, HBM 202B can receive a live recommendation 210B and predict the likelihood of user 214B adhering to the live recommendation 210B. In some embodiments HBM 202B can be a classifier type system which can embed psychometric data 220B, state data 216B, and a live recommendation 210B into a latent space. The live recommendation 210B can also be transmitted to a user 214B. In some embodiments, live recommendation 210B can be data transmitted to a user device, such as a portable electronic device, or the like.

Analyzer 208B can analyze a user adherence to the live recommendation 208B. In some embodiments, such analysis can include whether a user did or did not adhere to the recommendation, as well as the reasons why the decision was made. In some embodiments, a user application running on a user device can acquire such data.

A loss function 224 can measure a difference between a predicted user adherence generated by HBM 202B and an actual user adherence provided by analyzer 208B. Results (e.g., error values) provided by loss function 224 can be used by updater 206B to update HBM 202B.

While embodiments can include psychometric/behavior HBMs for arriving at recommendations with high probabilities of user adherence, such approaches can also be used for arriving at messaging approaches for a user.

FIG. 2C-0 is a block diagram of a system and method 200A according to further embodiments. A system 200A can include multiple HBMs 202-1 to -n. While HBMs (202-1 to -n) can be personalized HBMs, each trained with psychometric and response data particular to a user, HBMs (202-1 to -n0) can also be “phenotype” HBMs representing psychometric/behavior data of multiple users that has been subject to some classification or dimension reduction.

HBMs (202-1 to -n0) can be used to arrive at a messaging approach decision 226 for a user. A messaging approach decision 226 can be the selection of a particular messaging approach that has a high or highest likelihood of being effective on a per user basis. In some embodiments, a messaging approach can include the way in which choices can be presented to a user. In some embodiments, a messaging approach can include how a recommendation is provided to a user.

FIG. 2C-1 is a block diagram another system and methods 200B according to an embodiment. A system 200B can include an HBM 202B and training agent 228. In some embodiments, HBM model 202B can take the form of any of those shown in FIGS. 2A and 2B, including those initially seeded with psychometric data as well as those trained with user responses. An HBM model 202B can be trained with messaging approach types 232 and user data 230. User data 220 can include user responses as well as state data of a user. Such training can be based on acquired user data (previous user responses), live user responses and states, or a combination of both.

FIG. 2C-2 shows a system 2002 for executing inference operations with a trained HBM after it has been trained as shown in FIG. 2C-1. User data 230B for a new user (corresponding to training data 230) can be applied to trained HBM 234B to infer an optimal messaging approach 236 for that user. Such an optimal messaging approach can then be used by other systems (or other portions of system 200C, not shown) to generate messages aimed at a user.

Embodiments can also include methods and systems for generating quantifiable latent space representation of user lifestyles. Such a representation can help simplify behavior forecasting.

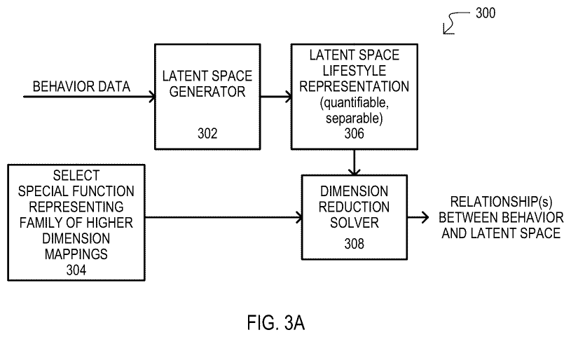

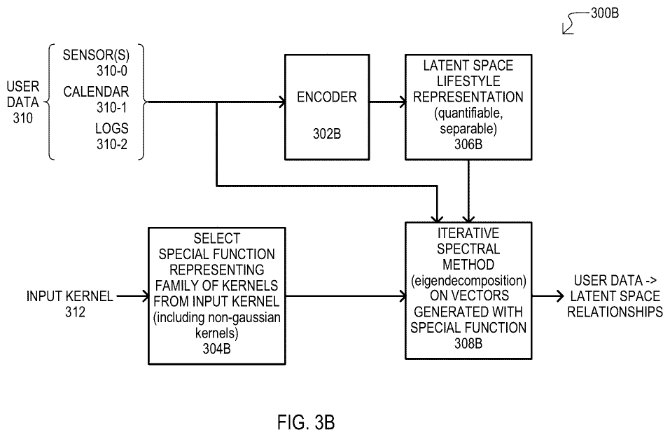

FIG. 3A is a diagram showing a system and corresponding method 300 according to another embodiment. A system 300 can include a latent space generator 302, function selector 304, and dimension reduction solver 308. A latent space generator 302 can map behavior data into a lower dimension to create a latent space lifestyle representation 306. A function selector 304 can select a function that represents a family of higher dimension mappings. In some embodiments, such a function can be one that represents a family of kernels. A dimension reduction solver 308 can utilize a selected function to capture relationships between behavior data and features of the latent space lifestyle representation 306.

FIG. 3B is a block diagram of a system and corresponding method 300B according to another embodiment. System 300B can include an encoder 302B, function selector 304B, and solver 308B. Encoder 302B can encode user data 310 into a latent space 306B. User data 310 can include any data suitable for representing a lifestyle. In the embodiment shown, user data 310 can include, but is not limited to, sensor data 310-0, calendar data 310-1 and behavior logs 310-2. An encoder 302B can include any suitable dimension reducer to map higher dimension user data 310 into lower dimension latent space 306B.

Function selector 304B can select a special function based on an input kernel 312. The special function can represent a family of kernels that includes the input kernel 312. A selected special function is not limited to families of Gaussian kernels and can include non-Gaussian kernels. A solver 308B can utilize an iterative spectral method 308B using the special function to determine relationships, including both linear and non-linear, between user data 310 and latent space 306B features.

Embodiments can also include methods and systems for recommending foods.

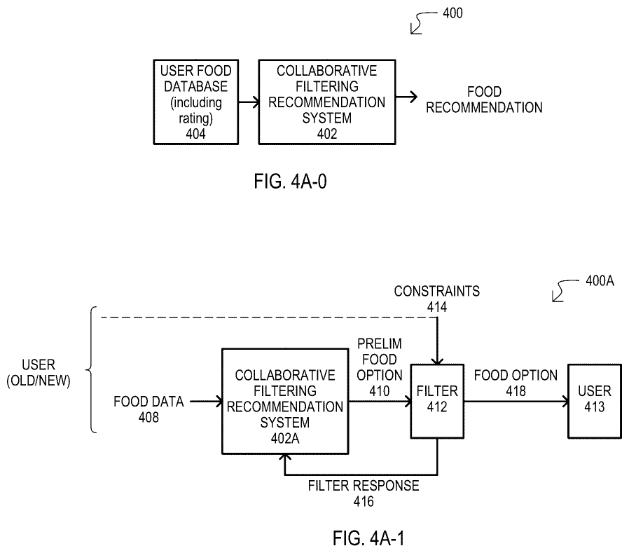

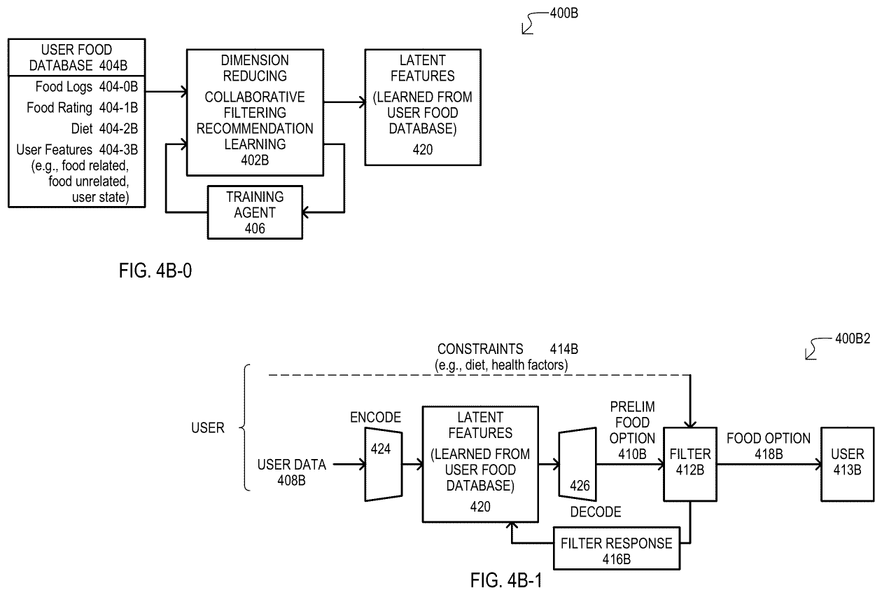

FIG. 4A-0 is a diagram showing a system and method for training a system to generate food recommendations by utilizing collaborative filtering. A system 400 can include a collaborative filtering recommendation system 402. Based on a user food database 404, recommendation system 402 can provide a food recommendation for a user. Such a recommendation can be based on correlation or similarity of choices as compared to other users. A recommendation system 402 can include an executed algorithm, machine learning system, or combination of both. A user food database 404 can include user food selections, and in some embodiments, can be a continuously updated database.

FIG. 4A-1 shows a system 400A according to another embodiment. A system 400A can generate food options 418 for a user 412 based on constraints specific to the user. Food data 408 from a user can be input to the recommendation system 402A which can output one or more preliminary food options 410. Food data 408 can be from an existing user (i.e., a user whose previous selections have been used to create recommendation system 402A), or a new user. Preliminary food options 410 can include nutritional information and/or other information related to food constraints for a user. Food options 410 can be selected or discarded with a filter 412 based on constraints 414. Any food options not eliminated by filter 412 can be provided to user 413. In some embodiments, a filter response 416 can be fed back to recommendation system 402A as filter response 416 to modify preliminary food options 410 further.

FIG. 4B-0 shows another system and method 400B according to an embodiment. A system 400B can include a dimension reducing collaborative recommendation system 402B. Collaborative recommendation system 402B can be trained to learn food user preferences with a user food database 404B. A user food database 404B can include, but is not limited to, food logs 404-0B, food ratings 404-1B, diet information 404-2B, and user features 404-3B. User features 404-3B can be food related and/or can be food unrelated, such as user states. In some embodiments, a recommendation system 402B can be trained with a training agent 406 and encode values of user food database 404B into a latent space 420. Latent space 420 may include latent features related to user choices not evident in only rating values.

FIG. 4B-1 shows a system and method 400B2 for generating food recommendations using a latent space like that shown as 420 in FIG. 4B-0. User data 408B can be encoded by an encoding operation 424 and applied to latent space 420 to generate encoded preliminary food options. Such encoded preliminary food options can be decoded into preliminary food options 410B. A filter 412B can filter preliminary food options 410B according to constraints 414B. Constraints 414B can be particular to a user (e.g., user corresponding to user data 408B). Constraints 414B can include, but are not limited to, diet and health factors for the user. After any filtering, a filter 412B can provide resulting food options 418B to a user 413B. In some embodiments, a filter response 416B can be provided which can modify latent space 420 in response to filter results.

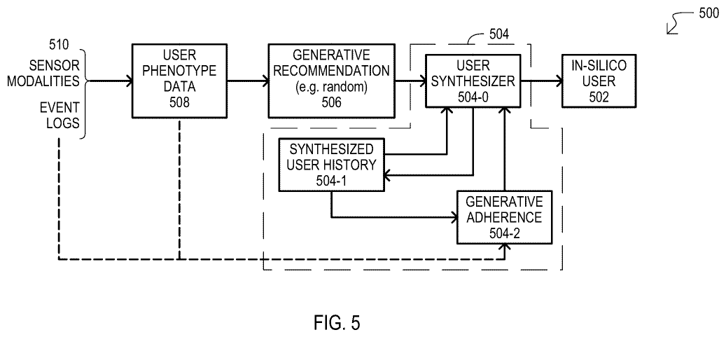

Embodiments can also include methods and systems for creating generative psychological adherence models of users. FIG. 5 is a diagram of a system (and corresponding method) 500 according to another embodiment. A system 500 can include a phenotype generator 508, a generative recommender 506, and a user synthesizer system 504. A phenotype generator 508 can receive user behavior data 510 data and generate user phenotype data. User phenotype data can organize users into groups having behavior commonalities. In some embodiments, phenotype generator 508 maps user behavior data 510 into a latent space. User behavior data 510 can include any suitable data that tracks a user behavior that is to be modelled. In some embodiments, user behavior data 508 can include data from different sensor types (modalities) and event logs that track user actions.

A generative recommender 506 can generate recommended actions for a user synthesizer system 504. In some embodiments, a generative recommender 506 can be seeded with user phenotype data 508. In some embodiments, generative recommender 506 can generate recommended actions in a random fashion.

A user synthesizer system 504 can include a user synthesizer 504-0, an adherence generator 504-2, and a synthesized user history 504-1. A user synthesizer 504 can be a model of user behavior that can be modified by training or the like. Adherence generator 504-2 can generate adherence responses to recommendations generated by generative recommender 506. In some embodiments, adherence generator 504-2 can determine adherence to a generated recommendation, at least in part, in response to user data, including user phenotype data 508 and/or user data 510. Responses (i.e., adherence) to generated recommendations can result in a synthesized user history 504-1.

After a user synthesizer system 504 is subject to multiple generated recommendations to create a synthesized user history 504-1, it can form an “in-silico” user 502. An in-silico user 502 can have various practical applications, including but not limited to serve as a model to predict human behavior or to train other models on human behavior.

Embodiments can also include methods and systems for generating simulated biophysical response signals that can be essentially indistinguishable from true biophysical signals.

FI6. 6A-0 is a diagram of a system (and corresponding method) 600A according to an embodiment. A system 600A can include a modifier system 602 that can receive “simple” predicted biophysical values 606 and generate realistic predicted biophysical values 610 therefrom. In the embodiment shown, the predicted biophysical values 610 can be blood glucose values, but alternate embodiments can include any other suitable biophysical value. A modifier system 602 can include a generative adversarial network (GAN) 604, which can be trained with actual biophysical response values 608. In some embodiments, this can include training a discriminator within GAN 604 with the actual biophysical response values 608.

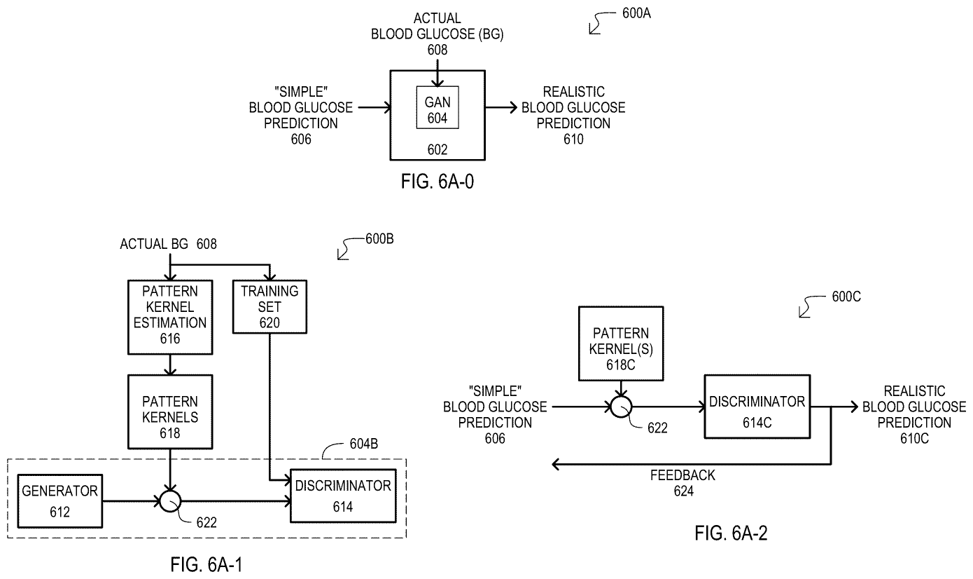

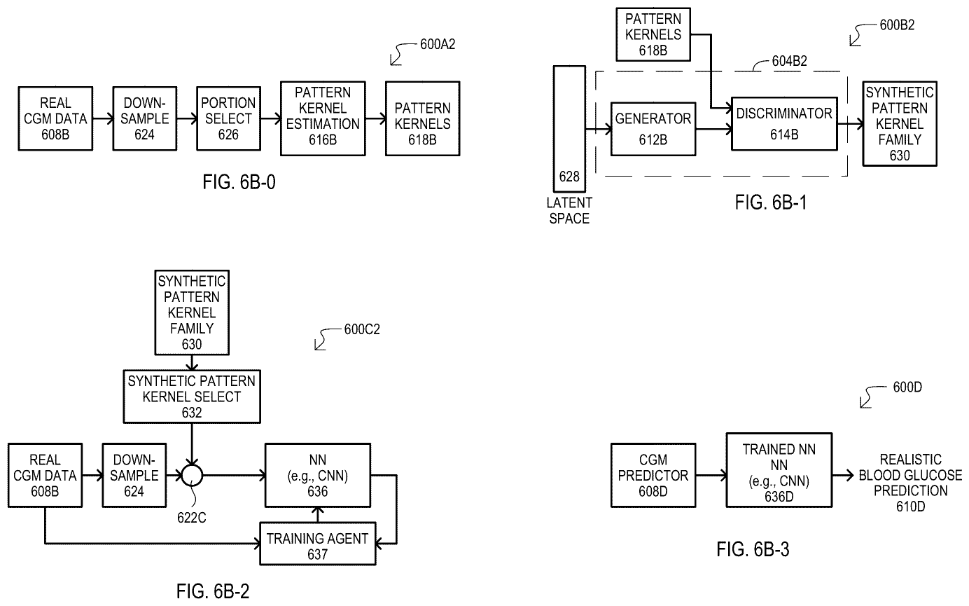

FIG. 6A-1 is a diagram of a system and method 600B according to another embodiment. A system 600B can train a GAN 604B to generate synthetic biophysical responses from actual biophysical response values 608.

A system 600B can include a kernel estimator 6A6 that receives actual biophysical response values 608 and can generate pattern kernels 618 therefrom. Pattern kernels 618 can represent variations in values that can occur in actual biophysical response.

Within a GAN 604B, a generator 612 can generate biophysical responses. Such generation can be essentially random or based on a seeded latent space. Generated biophysical responses can be combined 622 with a selected pattern kernel 618 to create a synthetic biophysical response which can be applied to a discriminator 614. A discriminator 614 can be trained with training data 620 to discriminate synthetic biophysical responses. Training data 620 can be derived from actual biophysical response values 608.

FIG. 6A-2 is a diagram of a system 600C for generating realistic predicted biophysical responses with a discriminator 614C trained as shown in FIG. 6A-1. A system 600C can include a “simple” predicted biophysical response values 606 that can be combined with pattern kernels 618C derived from actual biophysical response. A trained discriminator 614C can determine if a resulting predicted biophysical response is realistic enough. Those synthetic biophysical responses not rejected by trained discriminator 614C can be considered realistic blood glucose prediction values 610C. In some embodiments, results from trained discriminator 614C can be provided as feedback 624 for a system generating the simple biophysical response.

FIG. 6B-0 is a diagram of a system and method 600A2 for generating pattern kernels from real blood glucose (CGM) data. Such pattern kernels can be used to generate more realistic synthetic (e.g., predicted) CGM values. A system 600A2 can take real CGM data and execute a downsampling operation 624. Portions of the downsampled CGM data can be selected 626. As but one of many possible examples, portions having greater variations can be selected over portions of less variation. From the selected portion, a pattern kernel can be estimated 616B. Repeating this process on different sets of real CGM data can result in a collection of pattern kernels 618B.

FIG. 6B-1 is a diagram of a system 600B2 for generating a family of synthetic kernel patterns from real kernel patterns with a GAN 604B2. Within GAN 604B2, a generator 612B can generate potential synthetic kernel patterns from a latent space 628. A discriminator 614B can be trained with real kernel patterns 618B (which can be generated as shown in FIG. 6B-0) to evaluate synthetic kernel patterns. Those synthetic kernel patterns not rejected by discriminator 614B can form a synthetic pattern kernel family 630.

FIG. 6B-2 is a diagram showing how a neural network or other statistical model can be trained with a synthetic kernel pattern family 630 (which can be generated as shown in FI6. 6B-1). Real CGM data 608B can be downsampled 624. Such downsampling can be the same as that used in FIG. 6B-0 (i.e., that used to generate “real” pattern kernels from which synthetic pattern kernels can be created). A kernel selector 632 can select a synthetic kernel from a synthetic kernel pattern family 630. A synthetic pattern kernel selected by selector 632 can be combined with operation 622C with downsampled real CGM data 608B. In particular embodiments, a combining operation 622C can be convolution or some other suitable operation. A resulting value can be applied to model 636. Training agent 637 can update model 636 with the original real CGM data 608B. In some embodiments, a model 636 can be a CNN.

FIG. 6B-3 is a diagram showing a method and system 600D for the generation of more realistic predicted CGM values from initial predicted CGM values. A system 600D can include a trained model 636D, which can be a model as trained in FIG. 6B-2. A CGM predictor 608D can generate initial CGM values that are applied to model 636D which can generate realistic predicted CGM values 610D therefrom.

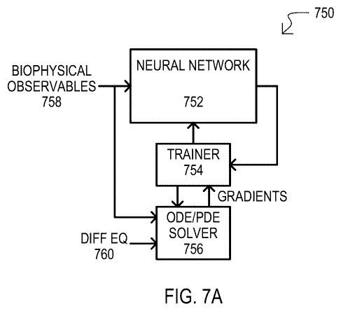

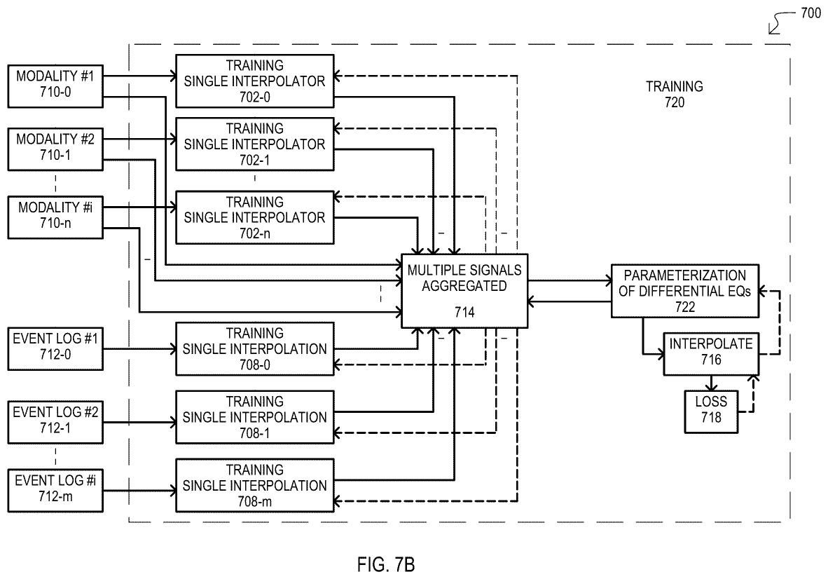

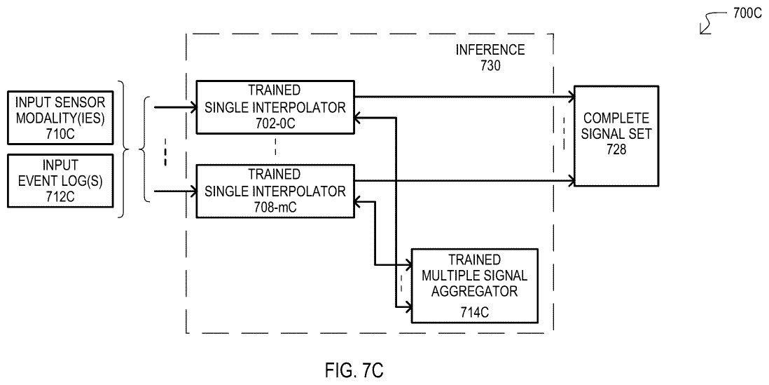

Embodiments can also include methods and systems for learning complex relationships between biophysical phenomena. FIG. 7A is a diagram of a system 750 according to an embodiment. A system 750 can include a neural network 752, training agent 754 and differential equation (DE) solver 756. A differential equation 760 can be provided to DE solver 756. A differential equation 760 can be an ordinary or partial differential equation, and DE solver 756 can be configured to solve the differential equation. A differential equation 760 can describe a general relationship between biophysical variables but lack parameters for accurate modeling.

A neural network 752 can undergo training by training agent 754 by using the DE solver 756. In particular, a neural network 752 can receive biophysical observables 758 which can express variables generally represented by the differential equation 760. Training agent 754 can utilize an error function that includes values from DE solver 756 to generate gradients for updating neural network 752.

Once trained, a neural network 752 can represent relationships contained within biophysical observables 758. In some embodiments, this can include parameters for the differential equation 760 that will enable accurate modeling of interrelated biophysical responses, including both linear and non-linear relationships.

In some embodiments, biophysical observables 758 can be time series data, and once trained, neural network 752 can be used to infer biophysical observables at various times. In some embodiments, a neural network 752 can create a latent space representation of biophysical relationships and can be used to infer or predict biophysical responses. The time series data may be activity data or food log data.

Embodiments can also include systems for interpolating multimodal sensory data. Such methods and systems can utilize differential equation modeling and solving as shown in FIG. 7A. A system and method according to such an embodiment is shown in FIGS. 7B and 7C. FIG. 7B shows the training of a system 700. FIG. 7C shows an inference operation of a system 700.

Referring to FIG. 7B, in a training operation 720, a system 700 can include a number of first single interpolators 702-0 to -n, second single interpolators 702-0 to -m, a signal aggregator 714, a parametizer 722, interpolator 716, and loss function 718. First single interpolators (702-0 to -n) can each receive sensor data of different sensor modalities 710-0 to -n and can be trained to interpolate values for their modalities. Sensor modalities (710-0 to -n) can represent different sensor values over a time period. Second single interpolators (708-0 to -m) can each receive event log data (712-0 to -m) representing different events experienced by users. Such events log data (712-0 to -m) can cover time periods also covered by sensor modalities (710-0 to -n). Second single interpolators (708-0 to -m) can be trained to interpolate values for event logs. Such training can include aggregated signal values generated by signal aggregator 714.

Signal aggregator 714 can aggregate multiple signals and event log data and provide aggregated values back to single interpolators (702-0 to -n, 708-0 to -m). In some embodiments, signal aggregator 714 can include a neural network or other statistical model. A signal aggregator 714 can map aggregated data into a latent space.

A parametizer 722 can generate parameters for one or more differential equation to train the output of signal aggregator 714. In some embodiments, such actions can include utilizing a differential equation solver in a loss function 718 as described herein and equivalents.

In some embodiments, training can include single interpolators (702-0 to -n, 708-0 to -m), in the absence of input values, generating data in response to values from signal aggregator 714. As but one example, while single interpolator 702-0 is not receiving sensor modality #1 data 710-0, single interpolator 702-0 can generate interpolated data values. Such training can occur on any of single interpolators (702-0 to -n, 708-0 to -m) as well as on multiples of single interpolators at the same time (to interpolate based on more than one missing data source of the aggregated data sources). While such training occurs, interpolator 716 can alter parameters for differential equations represented by multiple signal interpolator 714.

Referring to FIG. 7C, in an inference operation 730, some of trained single interpolators 702-0C to 708-mC can receive input signal data corresponding to the data on which they were trained, i.e., trained single signal interpolator 702-0C can receive inputs sensor data of modality #1 710C, trained single signal interpolator 708-mC can receive event log data of type #m 712C. However, one or more types of sensor modalities or event logs can be missing data. In such cases, the corresponding trained single interpolators will interpolate values for the missing data in response to outputs from trained signal aggregator 714C.

A resulting output from trained single interpolators (702-0C to 708-mC) can be a full set of data for all sensor modalities and/or full event logs, with interpolated data in place where data was missing.

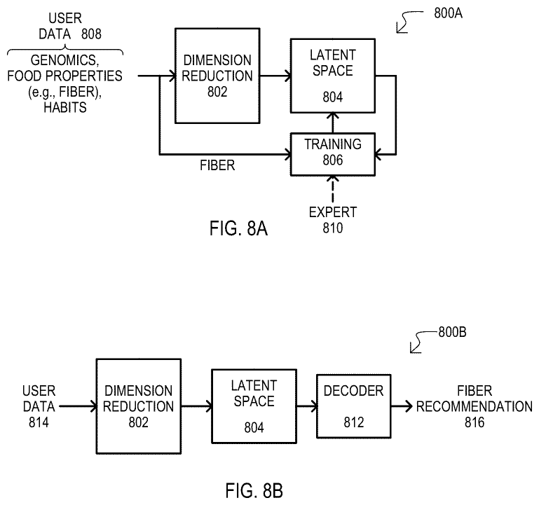

Embodiments can also include systems and methods for recommending foods having particular features based on user data. FIG. 8A is a diagram of a system and method 800A according to an embodiment. A system 800A can include a dimension reduction section 802 and a training agent 806. Dimension reduction section 802 can be trained to create a latent space 804 that can map user data 808 to a food selection that can maximize a particular food feature (in this case fiber content). It is noted that user data 808 can be data personalized to particular users, including but not limited to genomics data, foods eaten, and habits (dietary and/or non-dietary). In some embodiments, training can be supervised or partially supervised with expert data 810 on the desired feature (e.g., fiber).

FIG. 8B is a diagram of a system 800B for generating food recommendations according to an embodiment. User data 814 can be encoded by dimension reduction 802 into the latent space 804. Resulting mappings can be decoded 812 into one or more recommendations 816 that can maximize the desired feature (e.g., fiber).

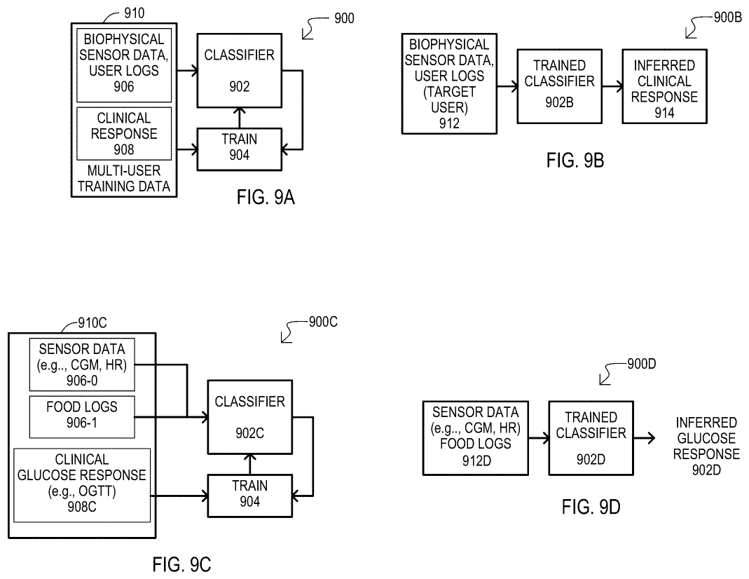

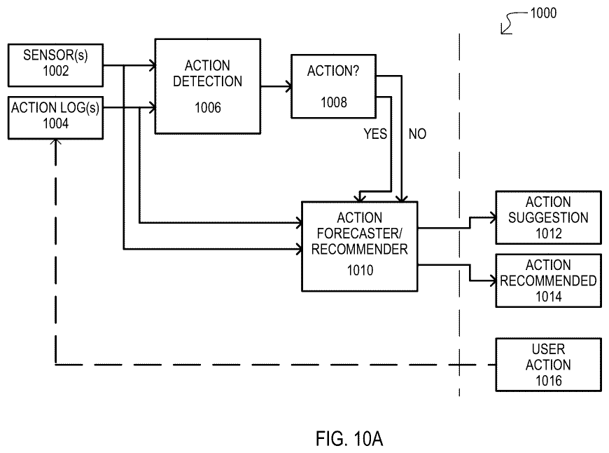

Embodiments can also include systems and methods for detecting diagnosable conditions based on sensor data and activity logs of a user. A system 900 according to an embodiment is shown in FIGS. 9A and 9B. FIG. 9A shows training of a system 900 according to an embodiment. A classifier 902 can be trained 904, via a training agent or the like, with biophysical sensor data and activity logs 906 of various users along with a corresponding clinical response 908 (i.e., a known clinical test to indicate a condition). Such training 904 can occur until classifier 902 can classify biophysical sensor data and activity logs 906 into corresponding clinical response 908.

FIG. 9B shows an inference operation of a system 900B according to an embodiment. Biophysical sensor data and user logs for a target user can be applied to a classifier 902B trained as shown in FIG. 9A. Trained classifier 902B can output an inferred clinical response 914, which can represent all or part of a diagnosable condition.

FIG. 9C is a block diagram of a system and method according to another embodiment for diagnosing pre-diabetes. Conventionally, pre-diabetes can be diagnosed with a hemoglobin A1C blood test which can be invasive and require a considerable amount of time. According to embodiments, systems can be trained with data from users having various glucose tolerances, including those with diagnosable states, such as pre-diabetes or type 2 diabetes, and but two examples. Using a target user's corresponding data, a glucose tolerance for the user can be inferred.

Referring to FIG. 9C, a system 900C can include a classifier 902C trained by a training agent 904 with user data 910C, which can include sensor data of users 906-0, including but not limited to, CGM data and heart rate (HR) data, food logs for such users 906-1, and the user's glucose response 908C. A glucose response 908C can be a diagnosis for the user, clinical results for a user (e.g., A1C test or oral glucose tolerance test), or a combination of both. Classifier 902C can be trained to classify sensor data 906-0 and food logs 906-1 into a clinical responses 902D.

FIG. 9D shows an inference operation of a system 900D according to an embodiment. Sensor data (e.g., CGM and HR) 912D can be input to trained classifier 902D, which can output an inferred glucose response 902D, which can include a probability of pre-diabetes.

Embodiments can also include systems and methods for determining when user actions have occurred but not been recorded or reported.

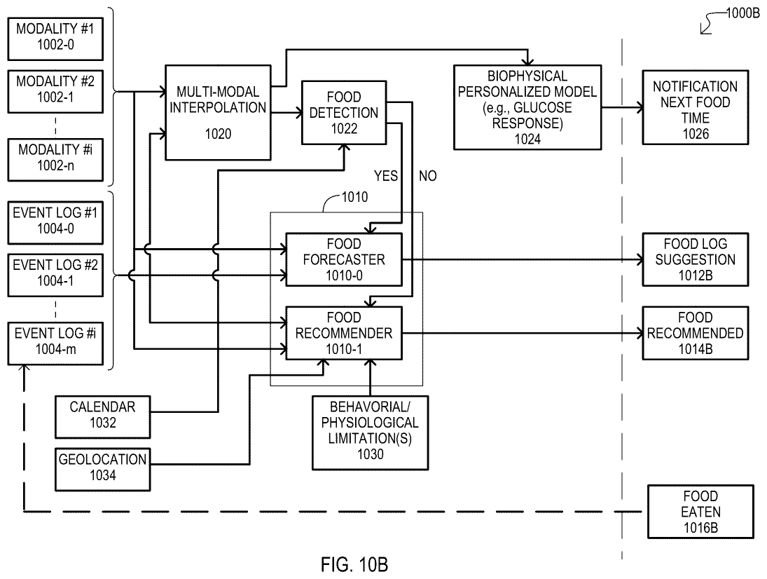

FIG. 10A is a diagram showing a system 1000 and corresponding method according to an embodiment. A system 1000 (and corresponding method) can include an action detector 1006, action determiner 1008, and an action forecaster/recommender (referred to hereafter as action forecaster) 1010. An action detector 1006 can receive sensor data 1002 for a user as well as action log data 1004 for the user. Action detector 1006 can determine when predetermined actions have been taken (or not taken) by a user based on such input data (1002, 1004). From such a determination, action detector 1006 can indicate whether the action was detected or not.

Action forecaster 1010 can be trained to forecast expected actions (which can be the same as those detectable by action detector 1006) based on the same input data (sensor data 1002, action log data 1004). If action forecaster 1010 forecasts an action and it is detected (Yes from action determiner 1008) and logged (recorded in action log 1004), no operation can occur by the action forecaster 1010. If action forecaster 1010 forecasts an action and it is detected but not logged (which can be determined by action determiner 1008), action forecaster 1010 can generate a suggested action 1012, which can be provided to a user for logging by the user.

If action forecaster 1010 forecasts an action and it is not detected and not logged (as can be determined by action determiner 1008), action forecaster 1010 can generate a recommended action 1014. A recommended action 1014 can be provided as a recommendation for a user. Such a recommended action can be generated based on data for a user (e.g., goals of a user).

In response to issuing an action suggestion 1012 or action recommendation 1014, a user can take an action 1016. Such an action can be reported back to a system 1000. In some embodiments, an action log 1004 can be updated with the user action 1016.

FIG. 10B is a diagram of a system 1000B and corresponding method according to another embodiment. A system 1000B include a multi-modal interpolation system 1020, food detector section 1022, food forecaster section 1010, and biophysical personalized model 1024. Sensor data of different modalities 1002-0 to 1002-n and event logs 1004-0 to 1004-m can be supplied to a multi-modal interpolator 1020. Multi-model interpolator 1020 can provide an output to biophysical personalized model 1024. Based on the biophysical personalized model 1024 of a user, a food notification 1026 for a future time can be generated that meets one or more nutrition goals of a user (e.g., glucose level).

Multi-modal interpolator 1020 can also provide outputs to food detector 1022. In the embodiment show, food detector 1022 can also receive calendar data 1032 of the user. Food detector 1022 can determine if food is eaten by a user. Food forecaster 1010 can include a forecast section 1010-0 and recommender section 1010-1.

Forecast section 1010-0 can forecast food selections based on user inputs (sensors modalities 1002-0 to -n, event logs 1004-0 to -m). If food is detected but not logged (which can be determined by food detector section 1022), forecast section 1010-0 can output a suggested food for logging 1012B. A food recommender 1010-1 can recommend foods based on user inputs (1002-0 to -n, 1004-0 to -m), geolocation data 1034, and limitations 1030 particular to a user. In some embodiments, a food recommender 1010-1 can recommend a food when food consumption is forecast but not detected. In the embodiment shown, food recommender 1010-1 can provide food recommendations 1014B.

Eaten food data 1016B by a user, such as that eaten or logged in response to suggestions or recommendations can be used to update a food log (1004-0 to -m).

### Computer Systems

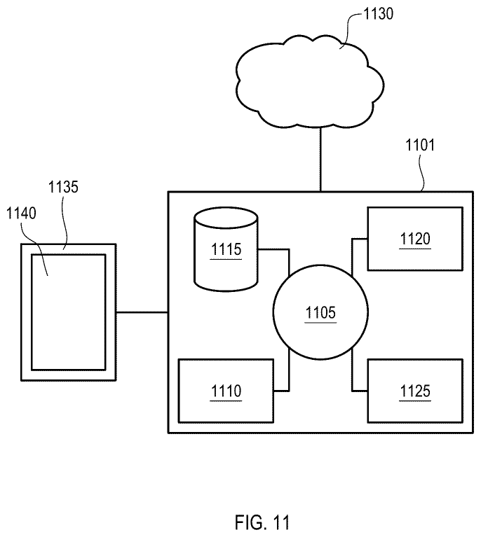

The present disclosure provides computer systems that are programmed to implement methods of the disclosure. FIG. 11 shows a computer system 1101 that is programmed or otherwise configured to perform biophysical modeling. The computer system 1101 can regulate various aspects of determining a biophysical model of the present disclosure, such as, for example, implementing machine learning algorithms. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a mobile computing device). Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as- compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, a window for viewing parameters of a biophysical model. Examples of UIs include, without limitation, a graphical user interface (GUI) and web-based user interface.

### Algorithms

Methods and systems of the present disclosure can be implemented by way of one or more algorithms. An algorithm can be implemented by way of software upon execution by the central processing unit 1105. The algorithm can, for example, generate an inferred biophysical response.

A method according to embodiment can include letting “T” be a number of time steps. A variable V can be a current most probable assignment to all T variables. In some embodiments, V can be initialized to be False for all variables. A value pV be the probability of the assignment V for a given T. A method can then, for a number of iterations i, from 1 to T: Compute the probability of modifying V by assigning True to each step in V, of which there are T steps, giving T assignments. * There is now the original V before this assignment and the T new V_t assignments. Let V for the next iteration be the old V, or one of the new V_t's with the highest probability. Let pV for the next iteration be the corresponding probability of the new V. V can then contain the most probable assignment to the random variables

On each iteration, one variable can be optionally converted from False to True, and the probability of the assignment after the iteration can only increases. On each iteration the variable that is set to true is the variable that, when set to True, most increases the probability of the entire assignment V, and thus the variable that most explains the currently unexplained input data. Since the effect of the variables on the input data is additive, as was assumed in the problem statement, the procedure will never assign True to a variable suboptimally, i.e. it would never need to back-track.

Using the meal example above, each iteration can pick a time step to optionally add one meal to the timeline, and that meal can be added in a way that most probably explains the blood glucose and other biomarker data.

In some embodiments, the evaluation of the probability of an assignment can be a joint probability over the T binary variables. In one embodiment, this joint probability could be constructed as the product of conditional probabilities using the law of conditional probabilities. For example, for T=3,

p(meal_1,meal_2,meal_3|input_data)=p(meal_3|meal_1,meal_2,input_data)*p(meal_2|meal_1,input_data)*p(meal_1|input_data).

In another embodiment, the conditional probability function could be a parameterized function where the parameters are learned using machine learning from a set of examples.

In another embodiment, the machine learned conditional probability function could be an artificial neural network.

In another embodiment, the machine learned, artificial neural network, conditional probability function could contain a recurrent neural network.

In another embodiment, the recurrent neural network could be an LSTM (long short-term memory) or GRU (gated recurrent unit).

In another embodiment, the machine learned, artificial neural network, conditional probability function could contain a transformer network.

In another embodiment, the inner evaluation of changing each of the variables to True can be performed in parallel on appropriately designed hardware accelerators such as GPUs (graphics processing units) or TPUs (tensor processing units). Such parallel computations can scale sub-linearly in the number of computations.

In another embodiment, the variables could be initialized to known True values. For example, when predicting meals, we may know the times of several meals before applying the described method. In this case, the proposed method returns the most probable assignment to the remaining unknown random variables.

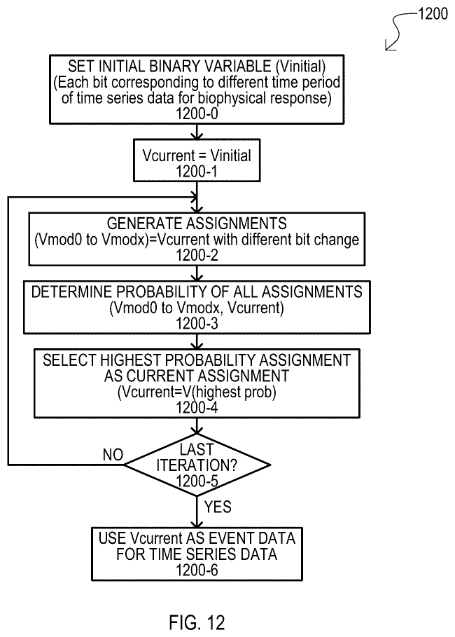

FIG. 12 is a flow diagram of a method 1200 according to another embodiment. A method 1200 can include setting an initial binary value 1200-0. Such an action can include setting each bit location of a multibit variable to a particular value, where each bit location corresponds to a time period of a corresponding time series data of a biophysical response. The value of each bit value of the assignment can correspond to the presence or absence of an event corresponding to the biophysical response. In some embodiments this can include setting all bit values to a same (e.g., invalid) state. However, alternate embodiments can have initial values with one or more locations set to a valid state (the event is known to have occurred at the particular time period).

A biophysical response can include any suitable response, including but not limited to blood glucose levels over time and/or heart rate over time. An event corresponding to the biophysical response can include a food (e.g., a meal) and/or physical activity. Such an initial value can be set to a current value 1200-1. A current value can be updated according to the method until a high (including highest) probability value is achieved.

A method 1200 can generate assignments 1200-2. Such an action can include generating variables (Vmod0 to Vmodx) that are each different from a current variable (Vcurrent) by one bit location being set to valid.

A probability for each variable, including Vcurrent, can be determined 1200-3. Such an action an include using a predetermined probability function that can take into account biophysical responses and the valid states of the variable. In some embodiments, such a function can be a machine learned function.

An assignment presenting the highest probability can be selected as the current variable (Vcurrent) 1200-4. If a last iteration has not been reached (NO from 1200-5), a method 1200 can repeat with a next set of assignments 1200-2. If a last iteration has been reached (YES from 1200-5), a method 1200 can utilize the current assignment (Vcurrent) as event data for the time series biophysical data 1200-6. A last iteration can occur when a higher probability than the current probability cannot be achieved.

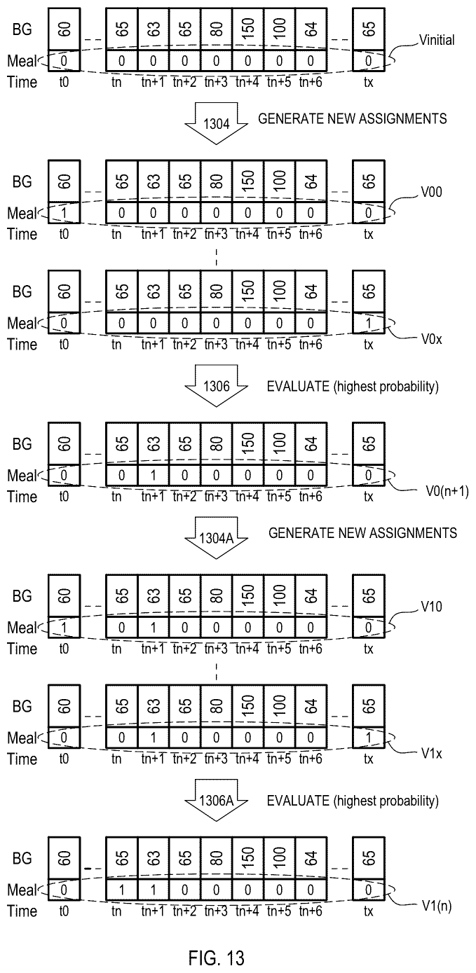

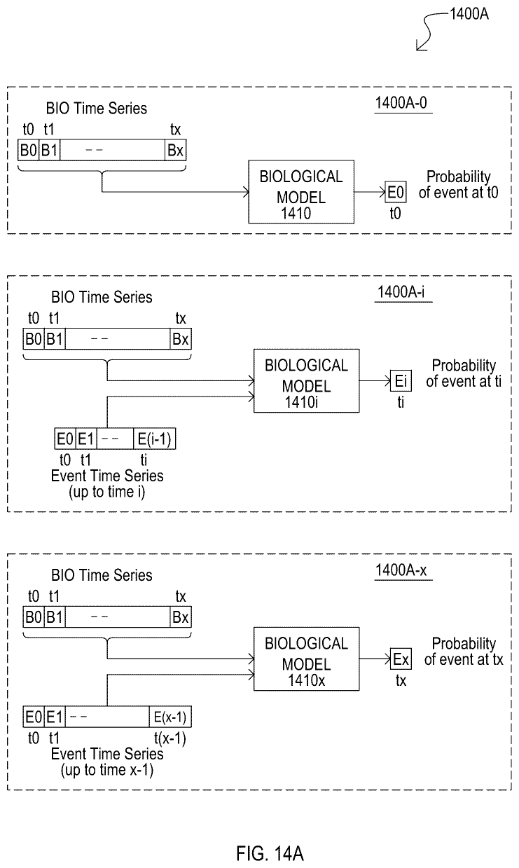

FIG. 13 is a sequence of diagrams showing variable generation and selection according to an embodiment. In FIG. 13, a variable is selected to arrive at meal events (Meal) corresponding to blood glucose time series data. (BG). Times for the time series data are shown as t0 to tx. The actions of FIG. 13 start at the top of the diagram and proceed downward.

An initial variable (Vinitial) can have all bit locations set to invalid (0 in this example). Assignments can then be generated 1304. Such an action can result in a set of variables (V00 to V0x), each of which has set one bit location set to active (1 in this example).

The variables (V00 to V0x and Vcurrent) can be evaluated for probability 1306 according to a probability function. While such a probability function can take any suitable form, in some embodiments such a probability function can be a conditional probability function as described herein, or equivalents.

A variable which yields the highest probability value can be selected as the new current variable. In the embodiment shown, variable V0(n+1) is selected has a highest probability function.

FIG. 13 shows a second assignment generation step 1304A, which generates a set of variables (V10 to V1x) according to a next iteration. The follow-on evaluation 1306A selects a highest probability variable V1(n) from the generated values (V10 to V1x) and the current highest probability variable (i.e., V0(n+1).

Such a process can continue until a higher probability variable cannot be achieved.

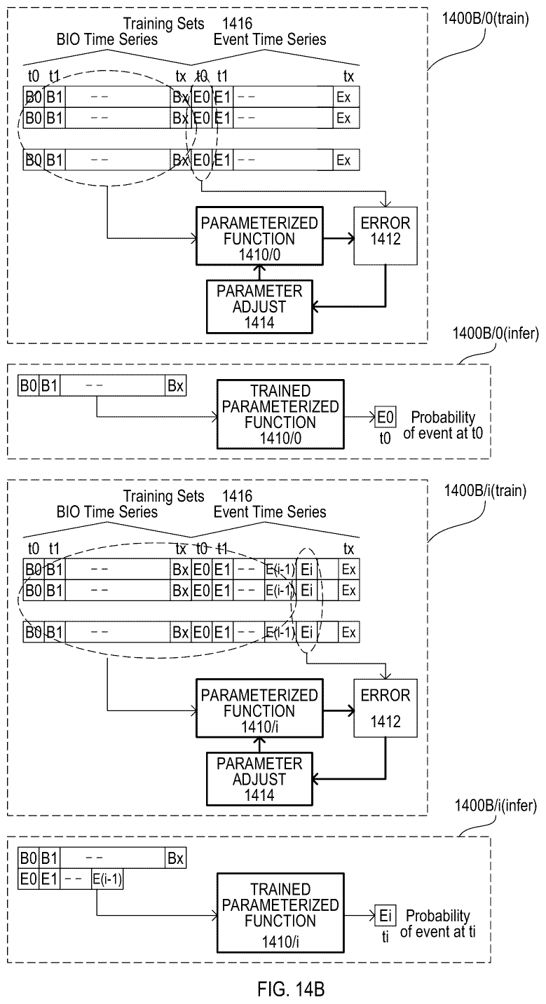

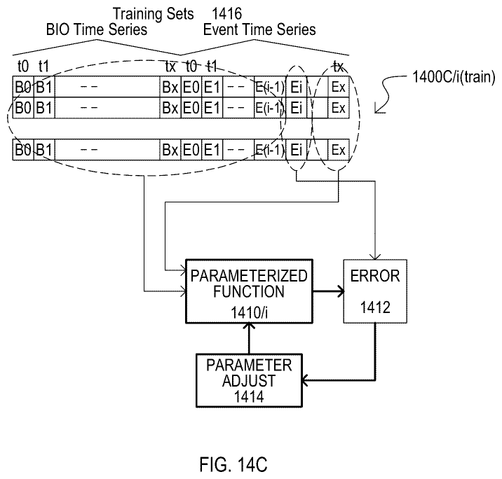

FIG. 14A shows a system and corresponding method 1400A for generating conditional probabilities according to an embodiments. A system 1400A can include a biological model that generates a probability for an event (e.g., meal) given time series biophysical data (e.g., blood glucose) B0 to Bx, and any previous event data.

Operation 1400A-0 shows a probability determination for an event at time t0 (E0). Such an operation can apply time series data B0 to Bx as inputs to model 1410 to generate the probability for an event at time to.

Operation 1400A-i shows a probability determination for an event at time ti (Ei). Such an operation can apply time series data (B0 to Bx) and event data leading up to time ti (E0 to E(i−1)) to model 1410i which can generate the probability of an event at time ti. A model 1410i may or may not be the same as model 1410.

Operation 1400A-x shows a probability determination for an event during a last time period (Ex). Such an operation can apply time series data (B0 to Bx) and event data leading up to time tx (E0 to E(x−1)) to model 1410x which can generate the probability of an event at time tx. A model 1410x may or may not be the same as model 1410 or 1410i.

It is understood that probabilities can be generated in this fashion for each event bit location (E0 to Ex), and such probabilities can be conditional probabilities which can be combined to generate an overall probability for the full event data (E0 to Ex).

FIG. 14B shows systems and methods for generating event data according to another embodiment. Training 1400B/0 (train) shows how a system can be trained with training data 1416 to infer the probability of an event at a first time period t0. A system 1400B/0 (train) can include a parameterized function 1410/0, an error function 1412 and a parameter adjust operation 1414. A parameterized function 1410/0 can be trained to infer the probability of event data for time to in response to biophysical time series data (B0 to Bx). Such training can include adjusting parameters of the function 1410 in response to error values. Error function 1412 can generate an error value by comparing data generated by function 1410/0 to corresponding event data at time t0 (E0).

System 1400B/0 (infer) shows a trained parameterized function 1410/0 (e.g., one trained shown for 1400B/0 (train)) that can receive biophysical time series input data (B0 to Bx) and generate a probability for an event at time to.

Referring still to FIG. 14B, training 1400B/i (train) shows how a system can be trained to infer the probability of an event at other time periods ti. A system 1400B/i (train) can include a parameterized function 1410/i (which may or may not be the same as 1400/0), an error function 1412 and a parameter adjust operation 1414. A parameterized function 1410/i can be trained to infer the probability of event data for time ti in response to biophysical time series data (B0 to Bx) and event data leading up to time ti (E0 to E(i−1)).

System 1400B/i (infer) shows a trained parameterized function 1410/i that can receive biophysical time series input data (B0 to Bx) and event data (E0 to E(i−1)) to generate a probability for an event at time ti.

While embodiments like that of FIG. 14B can train with date values the progress in time, alternate embodiments can include systems that train with all event data. FIG. 14C shows such an embodiment.

FIG. 14C shows training 1400C/i (train) which can create a function to infer the probability of an event at other time periods ti. A system 1400C/i (train) can include a parameterized function 1410/i. A parameterized function 1410/i can be trained to infer the probability of event data for time ti in response to biophysical time series data (B0 to Bx) and event data leading up to time ti (E0 to E(i−1) as well as that following time ti (E(i+1) to Ex)).

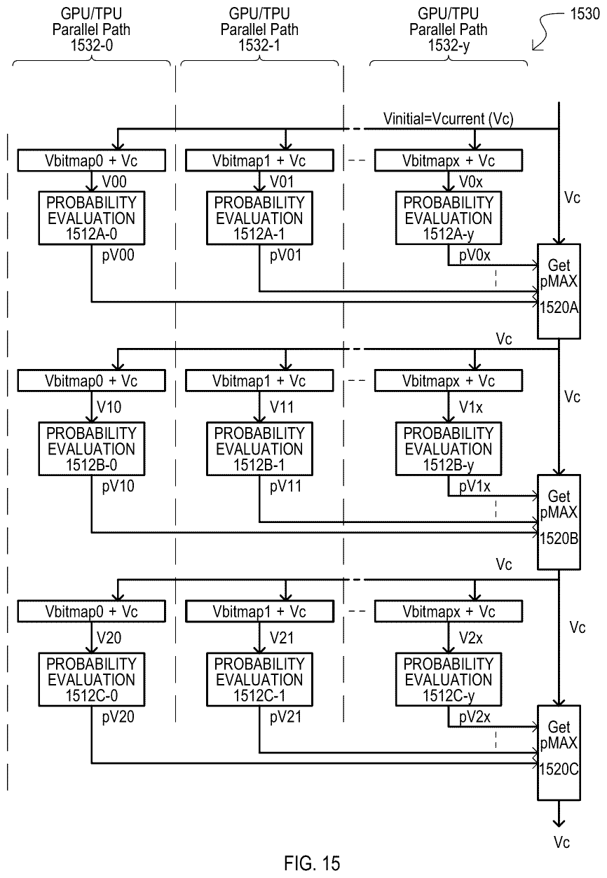

FIG. 15 is a diagram showing parallel processing operations of variable values according to an embodiment. Processing can occur in parallel for each bit change in a variable. Such processing can be executed with parallel processing capable hardware, such as a GPU and/or TPU. Each processing path 1532-0 to 1532-y can execute the same type of operation on different data values.

Each processing path (1532-0 to 1532-y) can set a different bit value in a current variable (Vcurrent=Vc) to true. Such an action can take any suitable form but is shown as a logical OR Vbitmapz+Vc, where z corresponds to the parallel processing path. Such an operation can generate variables for which probabilities can be calculated with a probability function (1512A/B/C-0 to 1512A/B/C-y). A highest probability variable can be selected as Vc 1520A/B/C for a next parallel operation.

Such operations can continue until a highest probability variable is reached.

Referring back to FIG. 1A, ML servers/systems 102 can include probability functions 1610. It is understood that probability functions 1610 are not necessarily machine learned functions.

In some embodiments, operations can utilize functions, models and/or data, including training data sets 1616, residing on data storage 122. In some embodiments, an application server 104 can forward data, such as time series biophysical data, to ML servers 1652.

In some embodiments, data storage 102 can store any of: models, function, parameters, or configuration data for configuring models/functions of ML systems 102.

In some embodiments, a data source 108 can correspond to one type sensor 116, which can be a continuous glucose monitor (CGM) that generates blood glucose time series data. Any other suitable sensors can be used to generate time series biophysical data, including a heart rate monitor 118. Further, as noted herein, time series data can be data logged by a user as well.

A subject device 1658 can be any suitable device, including but not limited to, a smart phone, personal computer, wearable device, or tablet computing device. A subject device 1658 can include one or more applications that can communicate with application server 1654 to provide data to, and receive data from, biophysical models residing on ML systems 1652. In some embodiments, a subject device 1658 can be an intermediary for any of data sources (1614-0, 1614-1).

The present disclosure provides methods for performing at least the following:
computing the most probable assignment of binary random variables to time series data using an iterative procedure that considers adding one new “True” value at every location on each iteration and retaining the most probable assignment from the current iteration to start the next iteration.
where the binary random variables represent meals being eaten at that time
where the time series consists of blood glucose measurements
where evaluation of the probability of a complete assignment makes use of an artificial neural network
where evaluation of the probability of a complete assignment makes use of a conditional probability distribution
where the conditional probability distribution is a recurrent neural network
where the conditional probability distribution is a transformer neural network
where the conditional probability distribution is a parameterized model whose parameters are learned from examples of time series data and the corresponding binary labels
where some of the binary random variables could be known before hand, constraining the result to produce assignments consistent with those known variables.

It is understood that various blocks shown in the figures described herein can include any of various circuits configured to execute the indicated functions, including but not limited to server systems that may or may not include customized hardware for accelerating operations, logic circuits, including custom logic circuits or programmable logic circuits. Such functions can also correspond to all or a portion of code executable by one or more processors that is stored on machine readable media. Data values as described herein can also be stored in machine readable media. Machine readable media can store code and/or data in a non-transitory form, in volatile and/or nonvolatile storage circuits.

It should be appreciated that in the foregoing description of exemplary embodiments of the invention, various features of the invention are sometimes grouped together in a single embodiment, figure, or description thereof for the purpose of streamlining the disclosure aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

It is also understood that the embodiments of the invention may be practiced in the absence of an element and/or step not specifically disclosed. That is, an inventive feature of the invention may be elimination of an element.

According to embodiments, blocks or actions that do not depend upon each other can be arranged or executed in parallel.

Accordingly, while the various aspects of the particular embodiments set forth herein have been described in detail, the present invention could be subject to various changes, substitutions, and alterations without departing from the spirit and scope of the invention.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method, comprising:
storing time series data with a storage device, the time series data including at least one biophysical response over sequential time periods;
by operation of a computing device
establishing an initial variable, having an event value corresponding to each time period,
generating a plurality of assigned variables, each having an assigned event value corresponding to each time period with one assigned event value being different with respect those of the initial variable and the other assigned variables,
evaluating the initial and assigned variables with a probability function to determine the initial or assigned variable having a highest probability of event occurrences with respect to the biophysical responses in the time periods, and
using the highest probability initial or assigned variable as the initial variable, repeating the generating the assigned variables and evaluating of the initial and assigned variables until a highest probability initial or assigned variable has been determined; and
using the highest probability initial or assigned variable to predict the at least one biophysical response in a user.

2. The method of claim 1, wherein the time series data comprises blood glucose values over the sequential time periods.

3. The method of claim 1, wherein the event value corresponds to a meal.

4. The method of claim 1, wherein the event value corresponds to physical activity

5. The method of claim 1, wherein:
the initial variable is a binary value, with each event value corresponding to a bit location of the binary value, each bit location corresponding to each time period of the time series; and
each assigned variable is a binary value, with each assigned event value corresponding to a bit location of the binary value, each bit location corresponding to each time period of the time series.

6. The method of claim 5, wherein:
establishing the initial variable includes setting all bits of the initial variable to a value indicating no event, and
generating the assigned variables includes changing a different bit in each assigned variable a value indicating an event.

7. The method of claim 1, wherein evaluating the initial and assigned variables with a probability function comprises applying the initial and assigned variables to a probability prediction statistical model to generate a probability value for each initial and assigned variable.

8. The method of claim 7, further including training a statistical model with training sets of time series data of the least one biophysical response and corresponding events to generate the probability prediction statistical model.

9. The method of claim 1, wherein evaluating the initial and assigned variables with the probability function includes determining a conditional probability for one time period using conditional probabilities for all previous time periods.

10. The method of claim 1, wherein:
the computing device comprises
a plurality of parallel processing paths, each processing path configured to determine a probability for a different initial or assigned variable, and
a variable selector coupled to each processing path and configured to select the highest probability initial or assigned variable.

11. The method of claim 10, wherein:
the computing device comprises a hardware accelerator unit selected from the group of: a graphics processing unit (GPU) and tensor processing unit (TPU); and
the parallel processing paths are within the GPU or TPU.

12. A system, comprising:
data storage configured to store time series data that included at least one biophysical response over sequential time periods; and
at least one computing device coupled to the data storage can configured to
establish an initial variable, having an event value corresponding to each time period of the time series,
generate a plurality of assigned variables, each having an assigned event value corresponding to each time period of the time series, one assigned event value being different with respect those of the initial variable and the other assigned variables;
evaluating the initial and assigned variables with a probability function to determine the initial or assigned variable having a highest probability of event occurrences with respect to the biophysical responses in the time periods,
using the highest probability initial or assigned variable as the initial variable, repeating the generating the assigned variables and evaluating of the initial and assigned variables until a highest probability initial or assigned variable has been determined, and
using the highest probability initial or assigned variable to predict the at least one biophysical response in a user.

13. The system of claim 12, wherein the at least one computing device comprises at least one statistical model configured to generate probability values for received initial variables and assigned variables.

14. The system of claim 13, wherein the at least one statistical model comprises at least one artificial neural network (ANN).

15. The system of claim 14, wherein the at least one ANN comprises a recurrent ANN.

16. The system of claim 15 wherein the at least one recurrent ANN is selected from the group of: a long short-term memory and gated current unit.

17. The system of claim 12, wherein the at least one computing device comprises at least one hardware accelerator unit having a plurality of processing paths, each processing path configured to determine the probability of the initial variable or one of the assigned variables.

18. The system of claim 17, wherein the at least one hardware accelerator is selected from the group of: a graphics processing unit and a tensor processing unit.

19. The system of claim 12, wherein the time series data comprises a blood glucose values over the sequential time periods.

20. The system of claim 19, wherein the event value is selected from the group of: a meal and a physical activity.
